(19) United States
(12) Patent Application Publication
(10) Pub. No.: US 2026/0363882 A1
(43) Pub. Date: Nov. 19, 2026

(54) Foamed Elastomer Prosthetic Liner

(71) Applicants: Aldo Laghi, Pinellas Park, FL (US); David Carson, Clearwater, FL (US); Jakob McClelland, Gulfport, FL (US); Nathaniel Vint, Palm Harbor, FL (US)

(72) Inventors: Aldo Laghi, Pinellas Park, FL (US); David Carson, Clearwater, FL (US); Jakob McClelland, Gulfport, FL (US); Nathaniel Vint, Palm Harbor, FL (US)

(73) Assignee: Alps South, LLC, St. Petersburg, FL (US)

(21) Appl. No.: 19/750,599

(22) Filed: Jul. 22, 2026

Related U.S. Application Data
(63) Continuation-in-part of application No. 18/661,095, filed on May 10, 2024.

(51) Int. Cl. A61F 2/78; A61F 2/50

(52) U.S. Cl. CPC A61F 2/7812; A61F 2002/5056; A61F 2002/785

(57) ABSTRACT

A prosthetic liner having a cylindrical or conical body with an open proximal end, a closed distal end, a posterior face, an anterior face, an internal thermoplastic elastomeric layer, and an external fabric layer, with a support material attached to the anterior face and a plurality of interior surface modifications on the internal thermoplastic elastomeric layer with the option of including at least one channel for wicking. The liner may be a locking or cushion liner. Also, the support material may be attached to the distal end of the liner inside of an enclosed pocket or inside of an open pocket attached to the exterior fabric layer. The support material is preferably a foam the foam selected from the group consisting of ethyl vinyl acetate, polyethylene, neoprene, ethylene propylene diene terpolymer, vinyl, silicone, and urethane. These liners allow for a bulbous formation at the distal end of a residual limb to give comfort to a user who may have a traumatic injury, scar tissue buildup, or simply seeking further comfort.

### CROSS-REFERENCE TO RELATED APPLICATIONS

This application is a continuation-in-part of, and claims the benefit of priority to, U.S. patent application Ser. No. 18/661,095, filed May 10, 2024, titled “Bonded Prosthetic Liner,” the entire disclosure of which is hereby incorporated herein by reference.

### BACKGROUND OF THE INVENTION

### Field of the Invention

The described invention relates to prosthetic liners. Specifically, the described invention relates to a prosthetic liner having varied supporting layers.

### Description of the Background Art

Prosthetic liners have been in use since the 1970's, mostly custom made, and made of various materials. Prosthetic liners serve the purpose of providing comfort for an amputee when wearing a prosthetic limb as the liner allows for a cushion between the residual limb and the socket supporting the prosthetic limb. Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb.

The liners disclosed in U.S. Pat. Nos. 5,443,525, 5,507,234, 5,728,168, 6,544,292, and 6,764,631 each represent advances in the field of fabric covered liners and the disclosures of which are hereby incorporated by reference. Additionally, U.S. Pat. No. 6,454,812, also hereby incorporated by reference, describes a liner comprising additional features attached to the liner textile to limit the vertical stretch at the distal end of a liner and is incorporated herein by reference. This method has been used successfully within the field of prosthetics to provide the liner textile with a strengthened distal end, means for attaching a threaded mechanical feature, and a boundary layer that prevents the thermoplastic elastomer from undesirably passing through the textile during high pressure processing.

The issue with each of these prior liners is that they lack the ability to incorporate padding, bladders, or other supporting material. This is because liners, as they are currently manufactured, either require injection molding or compression molding to bond the thermoplastic elastomeric layer to the exterior fabric layer. These processes generally require some form of pressure that is too great for supporting material to be reliably applied as the pressure causes the support material to deform.

Therefore, it is an object of this invention to provide an improvement which overcomes the aforementioned inadequacies of the prior art devices and provides an improvement which is a significant contribution to the advancement of the reinforced prosthetic liner art.

Another object of the invention is to provide a prosthetic liner formed using a textured mandrill creating surface modification of the interior thermoplastic elastomeric layer to allow for the addition of pads and other materials that would otherwise melt or become improperly configured due to the heat and/or pressure involved in standard molding processes.

Another object of the invention is to provide a prosthetic liner that allows for the inclusion of foam pads, spacer fabric, air bladders, fluid bladders, and other support materials.

Another object of the invention is to provide a prosthetic liner that allows for the introduction of electrical relays and sensors.

Another object of the invention is to provide a prosthetic liner that forms a composite material using heat and/or chemical bonding.

Another object of the invention is to provide a prosthetic that includes internal channels in the interior thermoplastic elastomeric layer that allows for the drainage of moisture.

Another object of the invention is to provide a prosthetic liner that includes therapeutic agents embedded in the interior thermoplastic elastomeric layer.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a prosthetic liner having a cylindrical or conical body with an open proximal end, a closed distal end, a posterior face, an anterior face, an internal thermoplastic elastomeric layer, and an external fabric layer, with a support material attached to the anterior face and a plurality of interior surface modifications on the internal thermoplastic elastomeric layer with the option of including at least one channel for wicking. The liner may be a locking or cushion liner. Also, the support material may be attached to the distal end of the liner inside of an enclosed pocket or inside of an open pocket attached to the exterior fabric layer. The support material is preferably a foam, the foam selected from the group consisting of ethyl vinyl acetate, polyethylene, neoprene, ethylene propylene diene terpolymer, vinyl, silicone, and urethane. These liners allow for a bulbous formation at the distal end of a residual limb to give comfort to a user who may have a traumatic injury, scar tissue buildup, or simply seeking further comfort.

In another embodiment, the support material comprises a foamed elastomer. The foamed elastomer is formed of an elastomeric material, such as a thermoplastic elastomer, silicone, or urethane, that has been augmented to include a plurality of voids, thereby providing augmented compression and shock-absorbing properties together with a reduced weight relative to the unfoamed elastomer. The voids may be introduced by a filler, such as glass bubbles, or by a blowing agent that foams the elastomer during processing through a thermal or chemical reaction. The percentage of voids may range from about 1%, providing a sturdy material suited for higher-use impact dissipation, to about 50% to 60%, providing a lighter weight and a highly cushioned, energy-dissipating pad. The foamed elastomer may form the entirety of the liner or may be provided as one or more targeted pads or relief areas, including multi-density pads, positioned at the tibial crest or the distal end of the liner.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a front perspective view of an embodiment of the present invention;

FIG. 2 is a cross-sectional view of the liner shown in FIG. 1 along line J-J;

FIG. 3 is a front perspective view of an alternative embodiment of the present invention;

FIG. 4 is a cross-sectional view of the liner shown in FIG. 3 along line L-L;

FIG. 5 is a cross-sectional view of the present invention with FIG. 5A showing a portion of the inner wall of the liner from element Q;

FIG. 6 is a front perspective view of an embodiment of the present invention with a support material adhered to the exterior fabric layer;

FIG. 7 is a cross-sectional view of the liner shown in FIG. 6 along line H-H;

FIG. 8 is a front perspective view of another embodiment of the present invention with a support material included as part of a removable pocket;

FIG. 9 is a cross-sectional view of the liner shown in FIG. 8 along line I-I;

FIG. 10 is a front perspective view of another embodiment of the present invention with a support material partially enclosed in a pocket;

FIG. 11 is a cross-sectional view of the liner shown in FIG. 10 along line K-K;

FIG. 12 is a front perspective view of another embodiment of the present invention with a support material included in an open anterior pocket and a closed distal pocket; and

FIG. 13 is a cross-section view of the liner shown in FIG. 12 along line L-L.

FIG. 14 is a front elevation view of another embodiment of the present invention in which the body of the liner is formed of a foamed elastomer;

FIG. 15 is an enlarged cross-sectional view of the distal end portion of the liner shown in FIG. 14, taken along line A-A, showing the fabric cover, the foamed elastomer, a reinforcement fabric, and a distal encapsulation;

FIG. 16 is a front elevation view of another embodiment of the present invention having a distal/anterior pad;

FIG. 17 is a cross-sectional view of the liner shown in FIG. 16, taken along line A-A; and18

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

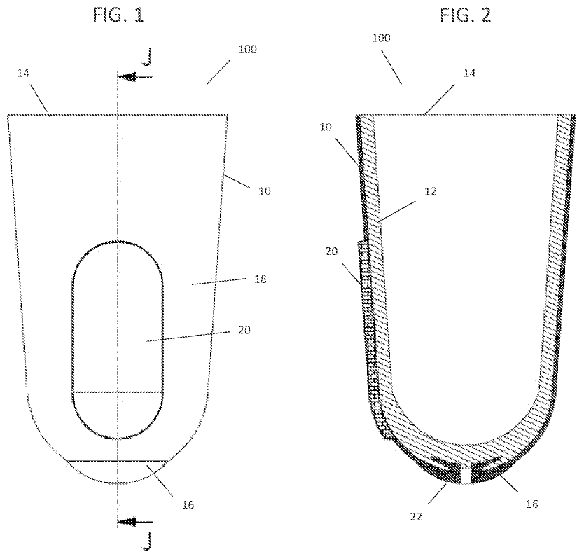

As shown in FIG. 1 and FIG. 2, one embodiment of the liner 100 is shown. The liner 100 generally has an external fabric layer 10 and an internal thermoplastic elastomeric layer 12 with an open proximal end 14 and a closed distal end 16. The internal thermoplastic elastomeric layer 12 is preferably a styrene-based thermoplastic elastomer such as SEEPS but may be any thermoplastic and elastomeric material comfortable on a user's skin, including silicone. The anterior face 18 of liner 100 includes a support material 20. Support material 20 can be adhered or sewn onto or into the external fabric layer 10 prior to bonding with the internal thermoplastic elastomeric layer 12 and can be a foam, spacer fabric (i.e. a multi-layered fabric with internal stitching allowing for a spring-like structure), an air bladder, a fluid bladder, or some other padded material that increases patient comfort. If adhered, a urethane-based adhesive is used on the external fabric layer 10 to prevent the fabric/support material combination from moving when the external fabric layer is being attached to the internal thermoplastic layer 12. The adhering process can utilize different adhesives (silicones, urethanes, etc.) or forgo adhesives all together by utilizing the thermoplastic properties and thermally bond the layers together. The support material 20 is preferably sized in relation to the size of the liner 100 and is preferably about ½ the width and ⅔ the height of the liner 100 but may be sized according to patient need. As seen in more detail in FIG. 2, one of embodiment of the liner 100 can also include a distal lock 22 which are common in prosthetic liners and known in the art.

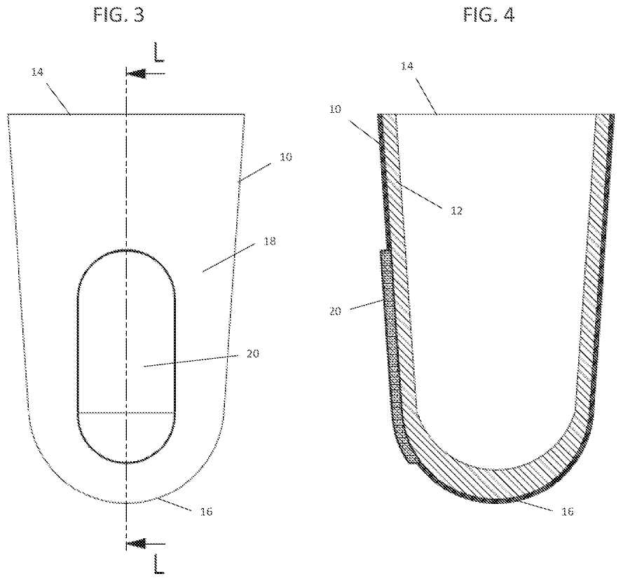

As shown in FIG. 3 and FIG. 4, another embodiment of the liner 100 does not need the distal lock 22 but is rather a suction-based liner. The liner 100 still incorporates an open proximal end 14, a closed distal end 16, an internal thermoplastic elastomeric layer 12, an external fabric layer 10, and a support material 20. For this particular embodiment, the support material 20 can also be placed on the distal end 16 as opposed to the anterior face 18.

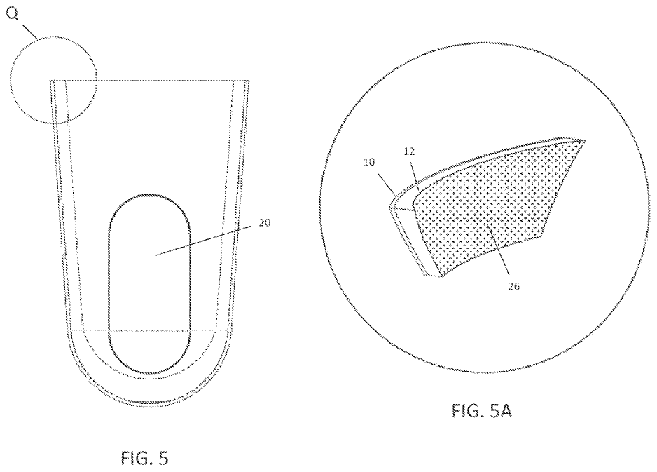

The internal surface 24 of the internal thermoplastic elastomeric layer 12 has interior surface modifications 26, as seen in FIG. 5A. The interior surface modifications 26 are useful by allowing the surface to grip a user's skin better thereby preventing movement and skin breakdown. The interior surface modifications 26 are effectively dimples throughout the internal thermoplastic elastomeric layer 12. Alternatively, the interior surface modifications 26 can include internal channels to allow moisture to drain down the surface to the distal end 16 (which may include a moisture port) or to wick upward towards the open proximal end 14. These internal channels may also be used for air which may become trapped during the donning procedure. The internal surface 24 may have different shapes or textures to create different effects. In addition, the internal surface 24 could have therapeutic agents such as antioxidant creams, antimicrobial agents, or pain management agents applied. This serves the benefit of having a known amount of whatever agent is used being applied to a user's skin as opposed to the potentially unknown amount caused by having the agent incorporated into the full thickness of the internal thermoplastic elastomeric layer 12.

The present invention can also incorporate electrical arrays or load bearing sensors that would normally be damaged through a typical molding and forming process. In another embodiment the internal thermoplastic elastomeric layer 12 could comprise a softer styrene-based gel on a posterior side and a denser styrene-based gel on the anterior side to protect the tibial crest. This allows for the creation of liners that have varying beneficial characteristics related to stretch, elongation, and general comfort. Incorporating the voids of a foamed elastomer allows for the elastomeric layer to be displaceable and compressible compounding the energy dissipation and increasing comfort. Skin health is also improved since the voids allow for more movement internally of the gel reducing the shear forces on the skin.

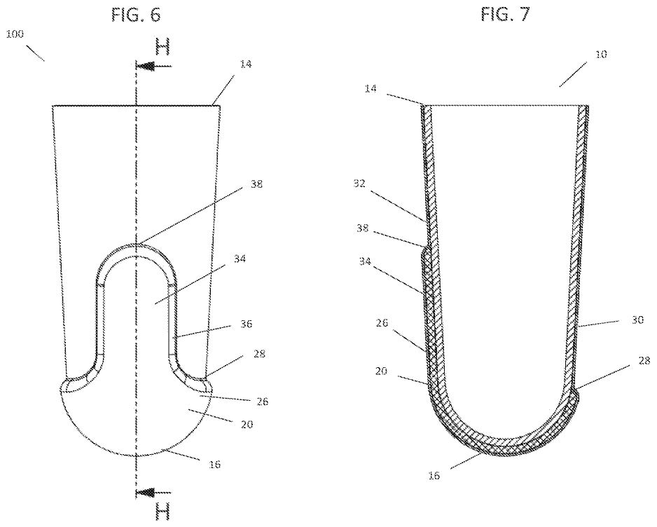

In situations where foam is used for the support layer 20, the support layer foam 26 can be open or closed-cell and should be compressible. As seen in FIGS. 6 and 7, support layer foam 26 is preferably placed distally because amputees having bony protrusions or nerve sensitivity will encounter those issues in those areas of the liner. Similarly, having padding distally is beneficial for amputees with high loading forces (i.e. high activity such as running, jumping, etc.) The support layer foam 26 can be made of a variety of materials such as ethyl vinyl acetate (EVA), polyethylene, an ionomer, neoprene, ethylene propylene diene terpolymer (EPDM), vinyl, silicone, or urethane. The support layer foam 26 may be cut into a variety of shapes. For example, as seen in FIGS. 6 and 7, the support layer foam 26 may circumferentially surround the closed distal end 16 which tapers to a circumferential end point 28 on the posterior side 30 of the liner 10 whereas on the anterior side 32 the support layer foam 26 extends upwardly towards the open proximal end 14 forming an anterior protrusion 34 which terminates at an anterior end point 38 which is prior to the open proximal end 14 and preferably about half of the length of the liner 10. However, it should be understood by those of ordinary skill in the art that a variety of shapes and configurations can be pursued based on patient need. The support layer foam 26 in this embodiment further includes a tapered edge 36 that allows the support layer foam 26 to have a thickness that ranges from ½ an inch at its thickest point to ⅛th of an inch at its narrowest.

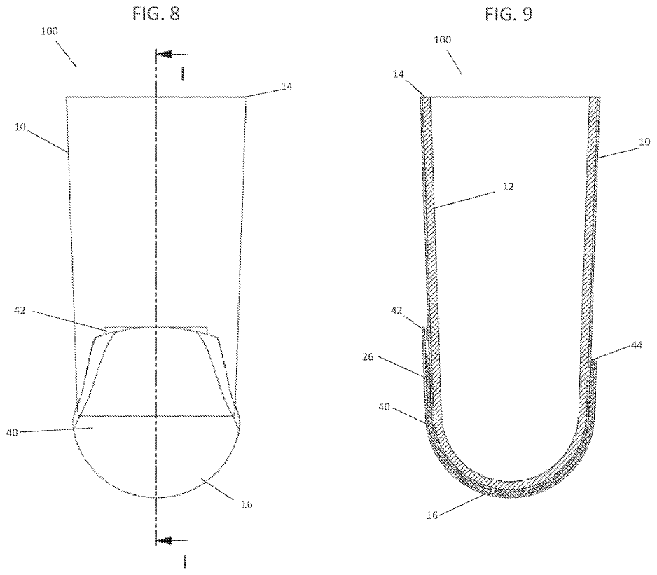

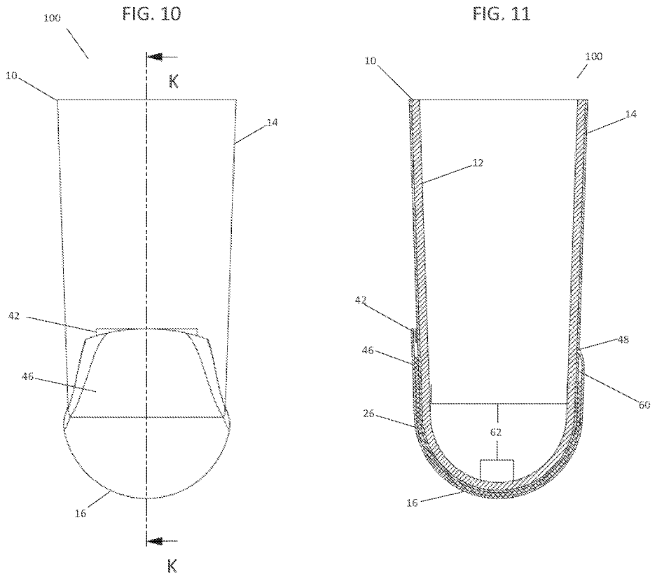

Alternatively, instead of having the support layer foam 26 included in the exterior fabric layer 10 prior to molding, the exterior fabric layer 10 of liner 100 may have an additional pocket added the exterior fabric layer 10 prior to molding over the internal thermoplastic elastomeric layer 12 as described above. As seen in FIG. 8 and FIG. 9, liner 100 may include a removable pocket 40 which is secured to the distal end 16 by an anterior hook and loop connection 42 and a posterior hook and loop connection 44. This setup allows the patient to add additional support layer foam 26 due to volume fluctuation or other comfort purposes or remove support layer foam 26 for similar reasons. Alternatively, as shown in FIG. 10 and FIG. 11, the support layer foam 26 can be inserted into a closed pocket 46 that is secured by an anterior hook and loop connection 42 and a closed anterior end 48. The support layer foam 26 does not necessarily have to fill the entirety of the cavity 60 formed by the closed pocket 46 when inserted into the closed pocket 46 but it should at least support the entire the curved region 62 of closed distal end 16.

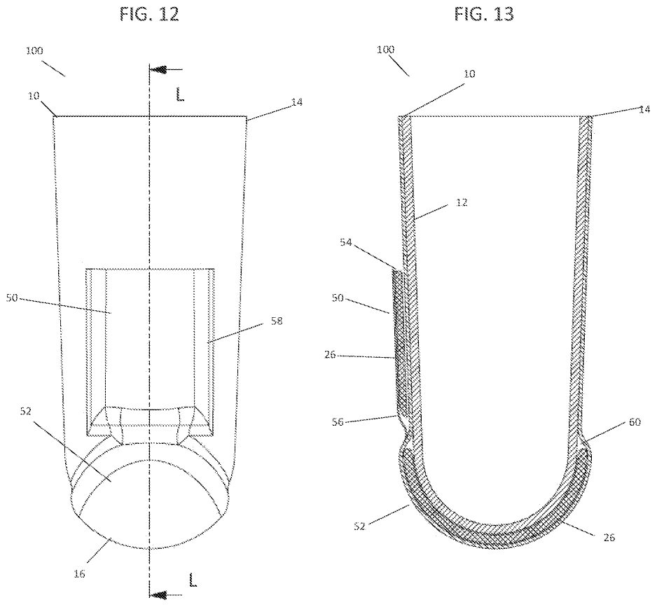

An additional alternative embodiment is shown in FIGS. 12 and 13. Here, the liner 100 includes two pocket styles for housing support layer foam 26 or other support material 20: an open anterior pocket 50 and a distal enclosed pocket 52. The open anterior pocket 50 has an open upper end 54 and a closed lower end 56 that tapers to the external fabric layer 10 at the closed lower end 56. The sidewalls 58 of open anterior pocket 50 also tapers to the external fabric layer 10. The distal enclosed pocket 52 is circumferential around the closed distal end 16 and contains support layer foam 26 or other support material 20 similar to the configuration shown in FIG. 6 and FIG. 7.

In another embodiment, the support material 20 comprises a foamed elastomer. Utilizing a foamed elastomer allows the support material 20 to be formed of a material similar to that of the internal thermoplastic elastomeric layer 12 while providing augmented material properties in compression and shock absorption, and further allows for a reduced weight because a percentage of the material is comprised of voids. The foamed elastomer may be a thermoplastic elastomer (TPE), silicone, or urethane that has been augmented by filling a percentage of the material with a filler, such as glass bubbles, or that has been augmented during manufacturing using a blowing agent that intentionally foams the material during processing to create voids within the material through a thermal or chemical reaction.

The foamed elastomer of support material 20 may be foamed to have a varying percentage of voids depending on the desired performance characteristics. For example, the foamed elastomer may be foamed to have a small amount of voids, such as about 1%, to provide a sturdy support material 20 suited for higher-use impact dissipation, or may be foamed to have a greater amount of voids, such as about 50% to about 60%, to reduce the weight of the support material 20 and to provide a very cushioned, energy-dissipating pad. In general, the percentage of voids may range from about 1% to about 60% of the material. Because the support material 20 may be formed of the same skin-friendly materials utilized elsewhere in the prosthetic liner, this embodiment allows the same materials to be utilized throughout the liner while affording total control over the shock-absorbing effects of the liner and lowering the overall weight of the liner.

The foamed elastomer may be incorporated into the liner 100 in a variety of configurations. In one configuration, the foamed elastomer is provided in pad form and is bonded to the liner 100, for example as the support material 20 described above with respect to the various embodiments and pockets. In another configuration, the foamed elastomer is a total-use material in which the entire product, including the full thickness of the liner 100, is formed of the foamed elastomer. In still another configuration, the foamed elastomer is utilized in one or more targeted pads or relief areas in which a separate density, or a multi-density pad, is provided at a particular location of the liner 100, such as the tibial crest or the closed distal end 16. The foamed elastomer may be used with shock-absorbing liners or with elastomer liners generally.

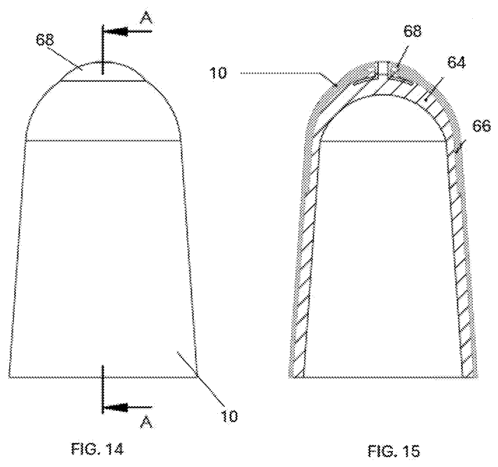

FIGS. 14 and 15 illustrate another embodiment of the liner 100 in which the body of the liner 100 is formed of a foamed elastomer 64. As best seen in the cross-sectional view of FIG. 15, the liner 100 in this embodiment includes an external fabric layer 10, also referred to as a fabric cover, disposed over the foamed elastomer 64, which forms the wall of the liner 100 and extends from the open proximal end 14 to the closed distal end 16. The foamed elastomer 64 may be any of the foamed elastomers described above, such as a thermoplastic elastomer, silicone, or urethane that has been augmented with a filler, such as glass bubbles, or with a blowing agent to introduce a plurality of voids ranging from about 1% to about 60% of the material by volume. Because the foamed elastomer 64 forms the full thickness of the wall of the liner 100, the liner 100 provides shock absorption and cushioning throughout its entirety while remaining lighter in weight than a comparable unfoamed liner.

As further shown in FIG. 15, the liner 100 includes a reinforcement fabric 66 disposed adjacent the internal surface 24 of the foamed elastomer 64 at or near the closed distal end 16. The reinforcement fabric 66 limits the vertical stretch of the liner 100 at the closed distal end 16, strengthens the closed distal end 16, and may provide a boundary layer that prevents the foamed elastomer 64 from undesirably passing through the external fabric layer 10 during processing. The closed distal end 16 further includes a distal encapsulation 68 in which the reinforcement fabric 66 is embedded within and encapsulated by the foamed elastomer 64 and/or an additional elastomeric material, thereby securing the reinforcement fabric 66 to the body of the liner 100 and providing a smooth, durable distal tip. The distal encapsulation 68 may also serve as a location for attaching a threaded mechanical feature, such as the distal lock 22, or another distal attachment.

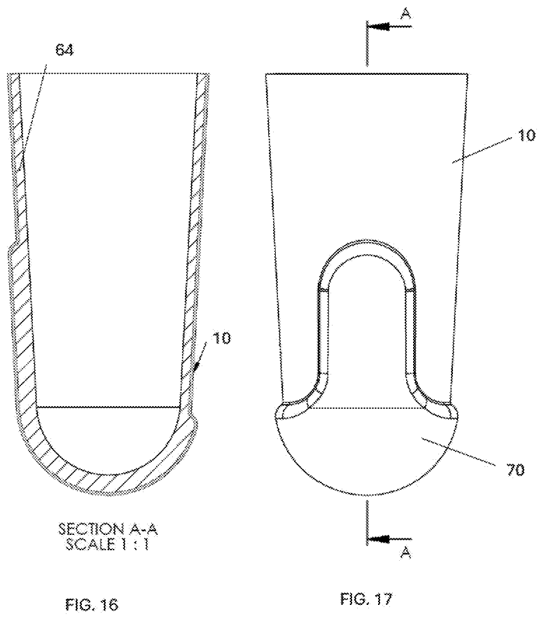

FIGS. 16 through 18 illustrate another embodiment of the liner 100 in which a distal/anterior pad 70 is provided. As shown in the front elevation view of FIG. 16 and the cross-sectional view of FIG. 17, the body of the liner 100 is again formed of a foamed elastomer 64 covered by an external fabric layer 10. The distal/anterior pad 70 is a pad of support material 20, preferably a foamed elastomer, that extends over the closed distal end 16 and upwardly along the anterior face 18 of the liner 100. The distal/anterior pad 70 is thickest at the closed distal end 16 and tapers as it extends proximally along the anterior face 18, providing additional cushioning and protection at the closed distal end 16 and along the anterior face 18 where an amputee is most likely to experience bony protrusions, nerve sensitivity, or high loading forces. The distal/anterior pad 70 may be a multi-density pad and may be provided in any of the pocket configurations described above with respect to FIGS. 6 through 13, or may be bonded directly to the liner 100.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A prosthetic liner comprising a body with an open proximal end, a closed distal end, a posterior face, an anterior face, an internal thermoplastic elastomeric layer, and an external fabric layer, and a support material attached to the body, wherein the support material comprises a foamed elastomer having a plurality of voids.

2. The prosthetic liner of claim 1 wherein the foamed elastomer is a material selected from the group consisting of a thermoplastic elastomer, silicone, and urethane.

3. The prosthetic liner of claim 1 wherein the plurality of voids comprises from about 1% to about 60% of the foamed elastomer by volume.

4. The prosthetic liner of claim 3 wherein the plurality of voids comprises about 1% of the foamed elastomer by volume to provide impact dissipation.

5. The prosthetic liner of claim 3 wherein the plurality of voids comprises from about 50% to about 60% of the foamed elastomer by volume to provide a reduced weight and an energy-dissipating pad.

6. The prosthetic liner of claim 1 wherein the plurality of voids is formed by a filler dispersed within the foamed elastomer.

7. The prosthetic liner of claim 6 wherein the filler comprises glass bubbles.

8. The prosthetic liner of claim 1 wherein the plurality of voids is formed by a blowing agent that foams the foamed elastomer during processing through one of a thermal reaction and a chemical reaction.

9. The prosthetic liner of claim 1 wherein the support material comprises a pad bonded to the body.

10. The prosthetic liner of claim 9 wherein the pad comprises a multi-density pad located at one of a tibial crest and the closed distal end of the body.

11. The prosthetic liner of claim 1 wherein the support material comprises a targeted relief area having a density separate from a density of the internal thermoplastic elastomeric layer.

12. A prosthetic liner comprising a body with an open proximal end, a closed distal end, an internal thermoplastic elastomeric layer, and an external fabric layer, wherein the internal thermoplastic elastomeric layer comprises a foamed elastomer extending throughout a thickness of the internal thermoplastic elastomeric layer, the foamed elastomer having a plurality of voids comprising from about 1% to about 60% of the foamed elastomer by volume.

13. The prosthetic liner of claim 12 wherein the foamed elastomer is a material selected from the group consisting of a thermoplastic elastomer, silicone, and urethane.

14. The prosthetic liner of claim 12 wherein the plurality of voids is formed by one of a filler dispersed within the foamed elastomer and a blowing agent that foams the foamed elastomer during processing.

15. The prosthetic liner of claim 12 further comprising a reinforcement fabric disposed adjacent an internal surface of the internal thermoplastic elastomeric layer at the closed distal end, the reinforcement fabric limiting vertical stretch of the liner at the closed distal end.

16. The prosthetic liner of claim 15 further comprising a distal encapsulation in which the reinforcement fabric is embedded within and encapsulated by the foamed elastomer at the closed distal end.

17. The prosthetic liner of claim 16 wherein the distal encapsulation provides a location for attaching a threaded mechanical feature.

18. The prosthetic liner of claim 9 wherein the pad comprises a distal/anterior pad extending over the closed distal end and upwardly along an anterior face of the body.

19. The prosthetic liner of claim 18 wherein the distal/anterior pad is thickest at the closed distal end and tapers as it extends proximally along the anterior face.

20. The prosthetic liner of claim 18 wherein the distal/anterior pad is a multi-density pad.
